(19) United States
(12) Patent Application Publication
(10) Pub. No.: US 2015/0366474 A1
(43) Pub. Date: Dec. 24, 2015

(54) DEVICES, SYSTEMS, AND METHODS FOR MONITORING BLOOD PRESSURE

(71) Applicant: THE CHARLOTTE-MECKLENBURG HOSPITAL AUTHORITY D/B/A CAROLINAS HEALTHCARE SYSTEM, Charlotte, NC (US)

(72) Inventors: David A. PEARSON, Charlotte, NC (US); Peter Thomas TKACIK, Fort Mill, SC (US); Jeffrey Allen KLINE, Carmel, IN (US)

(21) Appl. No.: 14/762,585

(22) PCT Filed: Jan. 22, 2014
(86) PCT No.: PCT/US2014/012515

§ 371 Date: Jul. 22, 2015

Related U.S. Application Data
(60) Provisional application No. 61/755,125, filed on Jan. 22, 2013.

(51) Int. Cl. A61B 5/022; A61B 8/00; A61B 8/06; A61B 5/00; A61B 5/026

(52) U.S. Cl. CPC A61B 5/02208; A61B 5/7282; A61B 5/7285; A61B 5/742; A61B 5/746; A61B 5/7415; A61B 5/026; A61B 5/6824; A61B 8/06; A61B 8/4488; A61B 8/4227; A61B 2562/04

(57) ABSTRACT

A system and method for measuring a subject's blood pressure is provided. The system includes a sphygmomanometer cuff configured to apply a pressure to a blood vessel region so as to restrict the flow of blood through a blood vessel of the region; at least one transducer configured to transmit a signal toward the region and to detect a return signal indicative of a velocity of blood flow through the blood vessel; and an apparatus having a processor configured to determine a systolic blood pressure of the subject. The processor is configured to determine whether the return signal corresponds to a first Korotkoff sound of the blood flow through the blood vessel, reduce pressure applied via the sphygmomanometer cuff if the return signal does not correspond to the first Korotkoff sound, and determine pressure applied via the sphygmomanometer cuff when the return signal corresponds to the first Korotkoff sound.

### FIELD OF THE INVENTION

The present invention relates generally to medical devices, systems, and methods for monitoring blood pressure. More specifically, methods and apparatuses are described for automatically measuring a patient's blood pressure on a near-continuous basis.

### BACKGROUND

Arterial blood pressure measurement is considered one of the most important diagnostic tools in medicine. Aside from being a possible indication of heart or endocrine problems, low blood pressure or hypotension (defined as an arterial systolic blood pressure of less than 90 mm Hg in adults), in particular, can be an indication of severe dehydration, blood loss, severe infection, or severe allergic reaction, among other things. Accurately measuring arterial blood pressure, especially in an acute setting such as an emergency room, can thus be essential to delivering effective treatment and assessing interventions.

In addition to the measurement itself, trends in a patient's blood pressure may also be indicative of a problem. Thus, although a single blood pressure reading may fall in a normal range, a comparison of that reading with a reading taken 5 minutes prior may show the start of a rapid decrease in pressure, which again may be an indication of a serious problem.

Accordingly, there is a need for devices, systems, and methods for accurately measuring blood pressure, even at low pressures, in a near-continuous manner.

### BRIEF SUMMARY OF EXAMPLE EMBODIMENTS

Accordingly, embodiments of systems, methods, medical devices, and computer program products for monitoring a subject's blood pressure on a continuous or near-continuous basis are provided. In one embodiment, a system for measuring a subject's blood pressure is provided that includes a sphygmomanometer cuff, at least one transducer, and an apparatus. The sphygmomanometer cuff may be configured to apply a pressure to a blood vessel region so as to restrict the flow of blood through a blood vessel of the blood vessel region. The at least one transducer may be configured to transmit a signal toward the blood vessel region and to detect a return signal indicative of a velocity of blood flow through the blood vessel. The apparatus may comprise a processor configured to receive the return signal detected by the transducer, determine whether the return signal corresponds to a first Korotkoff sound of the blood flow through the blood vessel, gradually reduce the pressure applied via the sphygmomanometer cuff in an instance in which the return signal does not correspond to the first Korotkoff sound, and determine the pressure applied via the sphygmomanometer cuff in an instance in which the return signal corresponds to the first Korotkoff sound. The pressure determined by the apparatus may be a systolic blood pressure of the subject.

The apparatus may be configured to automatically increase the pressure applied via the sphygmomanometer cuff to a pressure above the determined systolic blood pressure and gradually reduce the pressure applied via the sphygmomanometer cuff to re-determine the systolic blood pressure in a near-continuous blood pressure monitoring scenario.

In some cases, the system may comprise a plurality of transducers arranged in a staggered configuration. The apparatus may be configured to select one of the transducers corresponding to the highest strength of return signal so as to determine the systolic blood pressure via the selected transducer. Alternatively or additionally, the plurality of transducers may be configured to act as a phased array for determining a location of the blood vessel.

In some embodiments, the apparatus may further comprise a display, and the apparatus may be configured to present upon the display a graphical representation of the systolic blood pressure determined. The apparatus may be configured to provide an audible alarm in an instance in which the systolic blood pressure is outside a predetermined range of acceptable values.

In other embodiments, a medical device for measuring a subject's blood pressure is provided that includes a sphygmomanometer cuff configured to apply a pressure to a blood vessel region so as to restrict the flow of blood through a blood vessel of the blood vessel region and a plurality of transducers supported by the sphygmomanometer cuff, wherein each transducer is configured to transmit an ultrasonic signal toward the blood vessel region and to detect a return signal indicative of a velocity of blood flow through the blood vessel. The return signal detected by each transducer may be conveyed to an apparatus so as to allow a pressure applied via the sphygmomanometer cuff corresponding to a first Korotkoff sound detected via at least one of the plurality of transducers to be determined as a systolic blood pressure of the subject.

The plurality of transducers may comprise between 4 transducers and 10 transducers. Furthermore, the plurality of transducers may be arranged in a staggered configuration. In some cases, the plurality of transducers may comprise piezoelectric crystals, and the ultrasonic signal may have a frequency of between approximately 4 MHz and approximately 20 MHz. The plurality of transducers may be configured to act as a phased array for determining a location of the blood vessel.

In still other embodiments, an apparatus is provided that comprises at least one processor and at least one memory including computer program code. The at least one memory and the computer program code may be configured to, with the processor, cause the apparatus to at least receive a return signal detected by at least one transducer, wherein the return signal is indicative of a velocity of blood flow through a blood vessel. The at least one memory and the computer program code may be further configured to, with the processor, determine whether the return signal corresponds to a first Korotkoff sound of the blood flow through the blood vessel, gradually reduce the pressure applied via a sphygmomanometer cuff disposed proximate a subject's blood vessel region in an instance in which the return signal does not correspond to the first Korotkoff sound, and determine the pressure applied via the sphygmomanometer cuff in an instance in which the return signal corresponds to the first Korotkoff sound. The pressure determined by the apparatus may be a systolic blood pressure of the subject.

In some cases, the apparatus may be configured to automatically increase the pressure applied via the sphygmomanometer cuff to a pressure above the determined systolic blood pressure and gradually reduce the pressure applied via the sphygmomanometer cuff to re-determine the systolic blood pressure in a near-continuous blood pressure monitoring scenario. The apparatus may be configured to select one of a plurality of transducers corresponding to the highest strength of return signal so as to determine the systolic blood pressure via the selected transducer. Alternatively or additionally, the plurality of transducers may be configured to act as a phased array for determining a location of the blood vessel. In some embodiments, the apparatus may further comprise a display, and the apparatus may be configured to present upon the display a graphical representation of the systolic blood pressure determined. The apparatus may be configured to provide an audible alarm in an instance in which the systolic blood pressure is outside a predetermined range of acceptable values.

In still other embodiments, a method and a computer program product are described for monitoring a subject's blood pressure on a continuous or near-continuous basis by receiving a return signal detected by at least one transducer, wherein the return signal is indicative of a velocity of blood flow through a blood vessel; determining, via a processor, whether the return signal corresponds to a first Korotkoff sound of the blood flow through the blood vessel; gradually reducing the pressure applied via a sphygmomanometer cuff disposed proximate a subject's blood vessel region in an instance in which the return signal does not correspond to the first Korotkoff sound; and determining the pressure applied via the sphygmomanometer cuff in an instance in which the return signal corresponds to the first Korotkoff sound. The pressure determined by the apparatus may be a systolic blood pressure of the subject.

In some embodiments, the pressure applied via the sphygmomanometer cuff may be automatically increased to a pressure above the determined systolic blood pressure and gradually reduced to re-determine the systolic blood pressure in a near-continuous blood pressure monitoring scenario. In some cases, one of a plurality of transducers may be selected corresponding to the highest strength of return signal so as to determine the systolic blood pressure via the selected transducer. Alternatively or additionally, the plurality of transducers may be configured to act as a phased array for determining a location of the blood vessel.

In still other embodiments, a method and a computer program product are described for providing an audible indication of a quality of blood flow through a blood vessel comprising by determining a subject's blood pressure at a detected first Korotkoff sound; calibrating an audible output to a velocity of blood flow through the blood vessel at the determined blood pressure, wherein the audible output comprises a frequency component corresponding to the velocity of the blood flow through the blood vessel and an intensity component corresponding to the determined blood pressure at the detected first Korotkoff sound; detecting the velocity of blood flow through the blood vessel; extrapolating a blood pressure at the detected velocity; and adjusting the frequency and intensity components of the audible output in response to the detected velocity through the blood vessel at the extrapolated blood pressure. The subject's blood pressure may be re-determined at another detected first Korotkoff sound, and the audible output may be re-calibrated to a velocity of blood flow through the blood vessel at the re-determined blood pressure.

The first Korotkoff sound may be detected using a medical device comprising a sphygmomanometer cuff configured to apply a pressure to a blood vessel region so as to restrict the flow of blood through the blood vessel of the blood vessel region and a plurality of transducers supported by the sphygmomanometer cuff. Each transducer may be configured to transmit an ultrasonic signal toward the blood vessel region and to detect a return signal indicative of a velocity of blood flow through the blood vessel.

### BRIEF DESCRIPTION OF THE DRAWINGS

Having thus described the invention in general terms, reference will now be made to the accompanying drawings, which are not necessarily drawn to scale, and wherein:

FIG. 1 illustrates a system for monitoring blood pressure in accordance with an exemplary embodiment of the present invention;

FIG. 2 shows a schematic representation of an apparatus in communication with a pneumatic source and a device in accordance with an exemplary embodiment of the present invention;

FIG. 3 illustrates a display of an apparatus in accordance with an exemplary embodiment of the present invention;

FIG. 4 illustrates a sphygmomanometer cuff with mounted transducers in accordance with an exemplary embodiment of the present invention;

FIG. 5 illustrates transducers of the device of FIG. 1 transmitting and receiving signals in accordance with an exemplary embodiment of the present invention;

FIGS. 6A, 6B, 6C, and 6D illustrate different configurations of in accordance with an exemplary embodiment of the present invention; and

FIG. 7 illustrates a flowchart of methods of monitoring blood pressure in accordance with another example embodiment of the present invention.

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

Although each example described herein refers to measurement of arterial blood pressure via the brachial artery, embodiments of the described invention may be used to measure blood pressure via other arteries, such as the radial artery, the femoral artery, etc.

There are several ways that blood pressure can be measured according to conventional methods. The current initial standard for blood pressure measurements in most hospitals is via an automated non-invasive blood pressure device that uses oscillometric technology. This method correlates oscillations in the sphygmomanometer cuff pressure, which are caused by the oscillations of blood flow, with the patient's blood pressure. The sphygmomanometer cuff is placed around a patient's arm over the brachial artery location and is then automatically inflated and deflated. An electronic pressure sensor is used to detect the oscillations in cuff pressure, and the oscillations are then automatically interpreted. Although this method does not require as much skill to administer as other methods, the oscillometric technique does not provide continuous or near-continuous monitoring, is prone to delays in identifying hypotensive episodes (or may miss them all together), and may produce inaccurate readings or fail to register blood pressure due to reasons such as inaccurate cuff size or severe hypotension.

An alternative to oscillometric devices is the use of an intra-arterial catheter. This is an invasive method in which a cannula needle is placed in an artery, such as the radial, femoral, dorsalis pedis, or brachial artery. The cannula is in turn connected to a sterile, fluid-filled system, which is connected to an electronic pressure transducer. Although this method has the benefit of being both accurate and able to provide continuous monitoring, intra-arterial catheters are invasive, painful, technically difficult to insert, expensive, and often considered too time-intensive to apply, especially in an acute setting. Moreover, intra-arterial methods are associated with the risk of thrombosis, bleeding, infection, and neurovascular injury.

Yet another technique for measuring blood pressure is the standard Korotkoff method (auscultation method). Using this technique, a sphygmomanometer cuff is placed around the patient's brachial artery and inflated until the administrator (typically a nurse or doctor) listening with a stethoscope to the brachial artery at the elbow cannot hear the sound of blood through the artery. The administrator may then begin to slowly release the pressure in the cuff and listen for the sound of the blood as it just starts to flow in the artery. The sound has been described as a “whooshing” or pounding and is known as the first Korotkoff sound. The pressure at which the first Korotkoff sound is heard is considered the systolic blood pressure. The administrator can continue to listen to the arterial sounds as the cuff pressure is released, and the pressure at which the arterial sounds cease (known as the fifth Korotkoff sound) may be recorded as the diastolic arterial pressure. The Korotkoff method is considered the standard for blood pressure measurement; however, this method is prone to operator error, especially at low blood pressures and in environments where there may be loud background noises, such as in an emergency room setting. Moreover, the Korotkoff technique is unable to provide continuous or near-continuous blood pressure monitoring.

A modification on the Korotkoff method in which a hand-held Doppler device is used to “hear” the first Korotkoff sound is sometimes used to greatly reduce the risk of operator error. Although providing more accurate readings, even at lower pressures, this method still does not provide continuous or near-continuous readings, is moderately time-consuming, and requires expertise to administer.

By studying models of circulatory shock, the inventors have discovered that the conversion of the Doppler velocity measuring blood flow into continuous sound can provide clinically important information. For example, a decrease in blood flow velocity generally corresponds with a decrease in blood pressure. At the same time, the pitch, or audible component of the frequency of the sound waves caused by the blood flow, also decreases, and the phasic “snap” associated with systolic upstroke becomes more dull and vague. As described above, these sounds as perceived by an operator skilled in the art of resuscitation to audibly assess perfusion in an artery and thus predict the circulatory status of the subject being monitored. The inventors have discovered that the frequency of the same sound waves can be modulated and analyzed as representative of a corresponding pressure.

Moreover, the inventors have discovered that the quality of the sound waves (e.g., characteristics of the frequency, intensity, timbre, and/or harmonic regularity) may provide information about the velocity profile of the blood flow through the vessel being monitored. A velocity profile that is laminar may “sound” smooth, continuous, and clear, indicating a healthy blood pressure and/or healthy blood flow (e.g., unobstructed). In contrast, a velocity profile that is non-laminar (e.g., having turbulent eddies) may “sound” choppy, discontinuous, and/or dull, possibly indicating a critical situation of the subject. For example, at a Korotkoff sound corresponding to a cuff pressure of approximately 100 mm Hg, the sound produced by the blood flow may have a dominant frequency of between approximately 250 Hz to approximately 270 Hz and an intensity of approximately 65 dB. At a Korotkoff sound corresponding to a cuff pressure of approximately 60 mm Hg, however, the frequency of the sound produced by the blood flow may drop to between approximately 200 Hz to approximately 220 Hz and the intensity may increase to approximately 70 dB. The inventors have discovered that such components of the sound produced by the blood flow through the subject's blood vessel may be modulated and conveyed to a practitioner in the form of an audible output to convey information regarding the status of the subject without requiring the practitioner to look at a display or other visual output. In this way, the practitioner can be free to observe and attend to other matters while still obtaining vital information about the subject, which may be important especially in a setting of trauma resuscitation such as an emergency room of a hospital or a combat casualty care facility.

Accordingly, embodiments of the present invention provide devices, systems, and methods for automatically and accurately measuring a patient's blood pressure, even at hypotensive pressures, in a way that is non-invasive, continuous or near-continuous, and easy to administer.

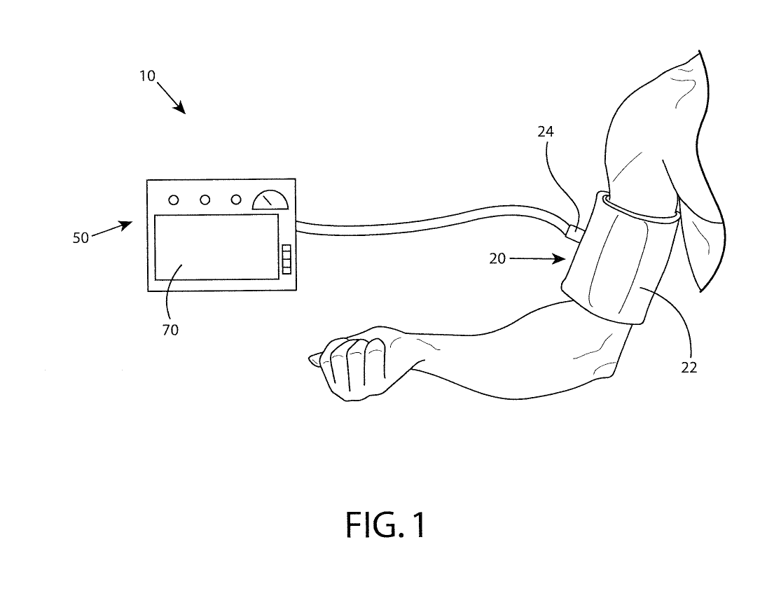

FIG. 1 depicts a system 10 configured for measuring a patient's blood pressure according to embodiments of the present invention. The system 10 includes a device 20 for measuring blood pressure and an apparatus 50 that is electrically connected to the device 20. For example, the device 20 may include a sphygmomanometer cuff 22 that is configured to apply a pressure to a blood vessel region so as to restrict the flow of blood through a blood vessel of the blood vessel region. For example, as shown, the sphygmomanometer cuff 22 may comprise an automatic valve 24 connecting the cuff to a pneumatic source (not shown), and the pneumatic source may be controlled by the apparatus 50 such that the apparatus can automatically inflate and deflate the cuff with air provided by the pneumatic source. The cuff may be configured to be placed around the subject's arm such that the flow of blood through the blood vessel, which in this example would be the brachial artery (e.g., in the arm), is affected by the pressurizing and depressurizing of the cuff.

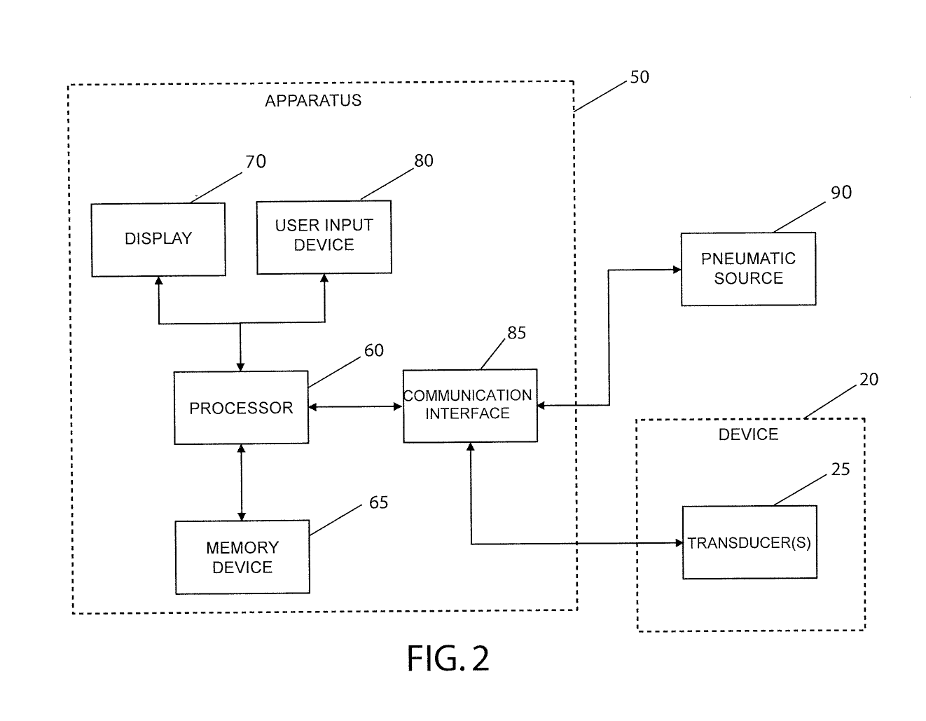

In some embodiments, the apparatus 50 may comprise a processor 60, as shown in FIG. 2, which controls the provision of signals to and the receipt of signals from various portions of the device 20 as well portions of the apparatus 50. In this regard, in some embodiments, the processor 60 may include circuitry desirable for implementing audio and logic functions of the apparatus 50. For example, the processor 60 may be comprised of a digital signal processor device, a microprocessor device, and various analog to digital converters, digital to analog converters, and other support circuits. The processor 60 may include functionality to operate one or more software programs, which may be stored in memory 65. For example, the processor 60 may be capable of operating software programs for filtering out noise from received signals, graphing trends in detected signal strength over time, comparing waveforms from detected signals to predefined waveforms, providing audible and visual alarms, analyzing data, executing algorithms, etc.

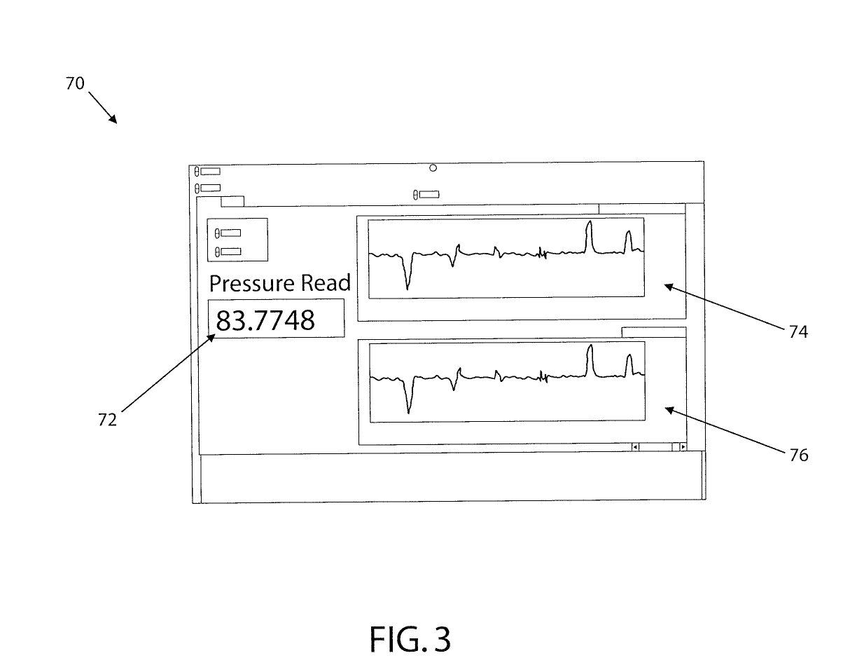

In some embodiments, the processor 60 may further be in communication with a display 70 that is configured to present images and/or textual data to a user. The apparatus 50 may, for example, be configured to present upon the display a graphical representation of the systolic blood pressure that is determined according to embodiments of the invention, as well as graphical representations of other physiological parameters that may be determined. FIG. 3, for example, depicts a display 70 providing a digital indication 72 of a subject's systolic blood pressure (83.7748 mm Hg) and graphical representations 74, 76 of the subject's normalized pressure and the Doppler signal showing a clear pulse.

The processor 60 may also be in communication with a user input device 80 of the apparatus, such as a keypad with hard and/or soft keys for operating the apparatus 50. In some cases, the display 70 may be a touch screen display that, in addition to being configured to display data to the user, is also configured to serve as the user input device by receiving touch inputs from the user via the display.

Also, as noted above, the apparatus 50 may include a memory device 65 in communication with the processor 60. The memory device 65 may include volatile memory, such as volatile Random Access Memory (RAM) including a cache area for the temporary storage of data. The apparatus 50 may also include other non-volatile memory, which may be embedded and/or may be removable. The memories may store any of a number of pieces of information, and data, used by the apparatus 50 to implement the functions of the apparatus as described herein. Additionally or alternatively, the memory device 65 could be configured to store instructions for execution by the processor 60.

In some embodiments, the processor 60 may further be in communication with a communication interface 85. The communication interface 85 may be a device or circuitry embodied in either hardware or a combination of hardware and software that is configured to receive and/or transmit data from/to a network and/or any other device or module in communication with the apparatus 50. In this regard, the communication interface 85 may include, for example, an antenna (or multiple antennas) and supporting hardware and/or software for enabling communications with a wireless communication network. In some environments, the communication interface 85 may alternatively or also support wired communication. As such, for example, the communication interface 85 may include a communication modem and/or other hardware/software for supporting communication via cable, digital subscriber line (DSL), universal serial bus (USB) or other mechanisms. The communication interface 85 may thus be configured to communicate with a pneumatic source 90 and/or one or more transducers 25 of the device 20 for allowing the processor 60 to direct the pressurization and depressurization of the sphygmomanometer cuff as well as to direct the receipt and transmission of ultrasonic signals via the transducers 25, as described in greater detail below.

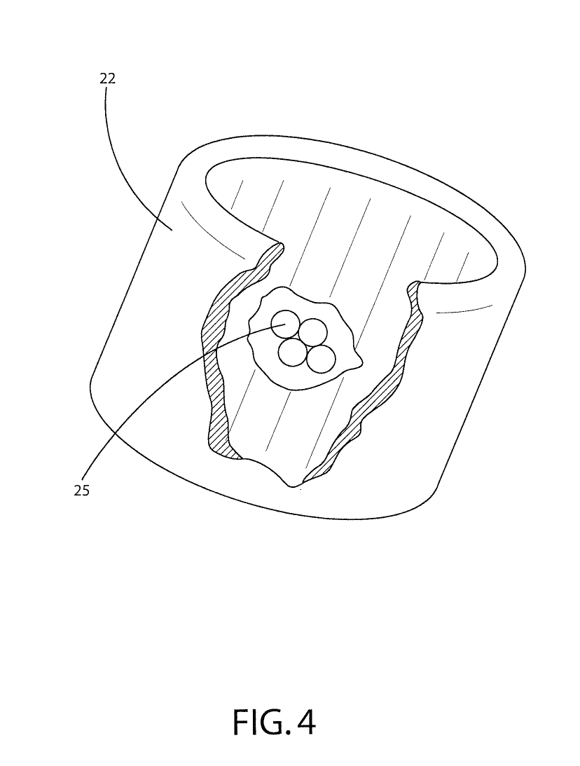

Turning again to FIG. 1, the sphygmomanometer cuff 22 may be configured, when pressure is applied via inflation by the pneumatic source, to stop the flow of blood through a blood vessel of the blood vessel region to which the cuff is applied (e.g., through the brachial artery in the upper arm region, as shown). One or more transducers 25 may be supported by the cuff 22, such as by being provided proximate an inner surface of the cuff, adjacent the subject's arm when the cuff is in place, as shown in FIG. 4. In other embodiments, the transducers may be mounted on a device that is separate from the sphygmomanometer cuff, such as on a ring, a patch, or other mounting accessory. The transducer device, in such a case, may be disposed downstream of the sphygmomanometer cuff, such as proximate the subject's elbow, on his or her forearm, or on his or her finger to detect the flow of blood through the blood vessel.

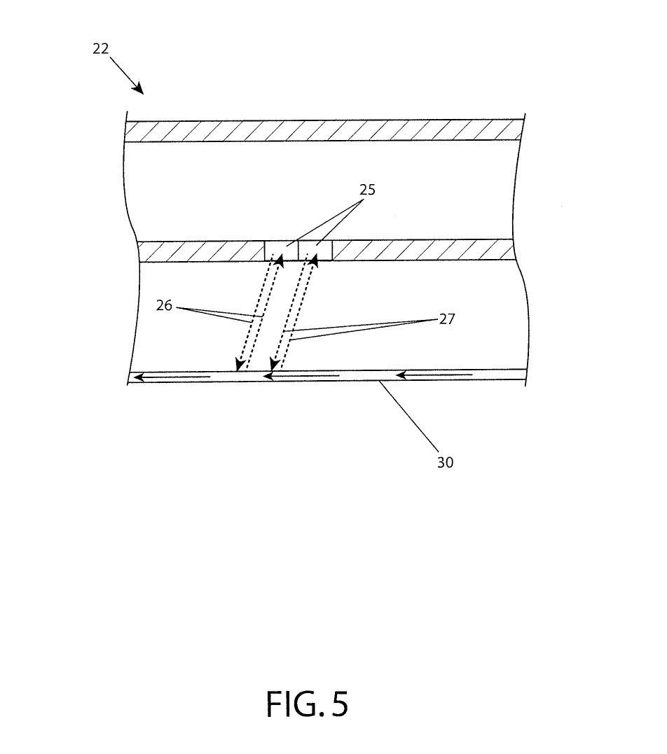

With reference to FIG. 5, each transducer 25 may be configured to transmit an ultrasonic signal 26 toward the blood vessel region (e.g., the subject's arm) and to detect a return signal 27 indicative of a velocity of blood flow through the blood vessel 30 (e.g., using Doppler principles). For example, an ultrasonic signal 26 may be transmitted at a frequency of between 4 MHz and approximately 20 MHz, and the frequency of the return signal 27 may be detected and conveyed to the processor 60 (e.g., via the communication interface 85 of the apparatus 50 shown in FIG. 2). By analyzing the frequency of the return signal 27 as compared to that of the original ultrasonic signal 26, the apparatus 50 (e.g., via the processor 60) may be able to determine whether blood is flowing through the blood vessel 30.

The frequency of the ultrasonic signal 26 may be selected to achieve an appropriate depth of penetration of the signal (e.g., projection of the signal toward the blood vessel) with respect to an area that is covered by the signal (e.g., size of the region on the subject's arm covered), while at the same time achieve a clear signal that is relatively free of noise. An ultrasonic signal with a frequency of 4 MHz, for example, may have a wide field of coverage, but the signal itself may be too noisy to result in clear and useful return signals that are indicative of the first Korotkoff sound. In contrast, however, an 8 MHz signal may have a field of coverage measuring only 3 mm×6 mm in diameter (e.g., a pinpoint), but may provide a clean and crisp signal that is free of most noise and can easily produce return signals useful for determining the subject's blood pressure. Moreover, any additional filtering that is necessary may be accomplished via filtering software at the processor.

According to some embodiments, when pressure is initially applied to the blood vessel 30 via the sphygmomanometer cuff 22, the pressure is high enough that the blood vessel is effectively closed off and there is no or minimal blood flow through the vessel. In this case, the return signals 27 that are detected indicate no or very slow blood flow through the blood vessel 30. In response to this determination, the processor 60 is configured to direct the sphygmomanometer cuff 22 (e.g., via an automatic valve) to gradually reduce the pressure being applied to the blood vessel and blood vessel region. For example, the pressure applied by the sphygmomanometer cuff 22 may be decreased from 140 mm Hg to 135 mm Hg, then from 135 mm Hg to 130 mm Hg, then from 130 mm Hg to 125 mm Hg, and so on until the return signal 27 that is detected corresponds to the first Korotkoff sound, indicating that blood has begun flowing through the blood vessel 30. In an instance in which the return signal 27 corresponds to the first Korotkoff sound, the pressure being applied to the blood vessel region may be determined by the apparatus 50. This pressure may then be considered the subject's systolic blood pressure.

In some embodiments, the apparatus 50 may be configured to operate in a near-continuous blood pressure monitoring scenario. For example, once the subject's systolic blood pressure has been determined as described above, the apparatus 50 may be configured to automatically increase the pressure applied via the sphygmomanometer cuff 22 to a pressure above the determined systolic blood pressure, such as by directing an automatic valve on the sphygmomanometer cuff to open and directing the pneumatic source 90 (FIG. 2) to re-inflate the cuff to a given pressure. In an example in which the systolic blood pressure is determined to be 83 mm Hg, for example, the cuff may be inflated to a pressure of between 93 mm Hg and 98 mm Hg to ensure that the blood flow through the blood vessel is stopped. Then, as described above, the pressure applied via the cuff may be gradually reduced once more to allow the apparatus to re-determine the systolic blood pressure. The process of inflating and deflating the sphygmomanometer cuff 22 may be done relatively quickly, such that a new systolic blood pressure reading is taken every 15-20 seconds, providing near-continuous monitoring of the subject's blood pressure. Moreover, rather than completely deflate the sphygmomanometer cuff 22 with each cycle once the blood pressure is determined, the cuff may only be deflated to a certain predefined pressure with respect to the systolic pressure that is determined. Thus, for the example of a systolic reading of 83 mm Hg, the cuff may be inflated to 95 mm Hg and may be deflated to a pressure of 70 mm Hg before starting the cycle over again. The apparatus 50 may further be configured to automatically cease the near-continuous blood pressure monitoring scenario after a predetermined amount of time has passed, such as after the passage of 15 minutes to 25 minutes for the safety of the subject (e.g., to avoid tourniquet issues associated with prolonged application of pressure to the blood vessel region).

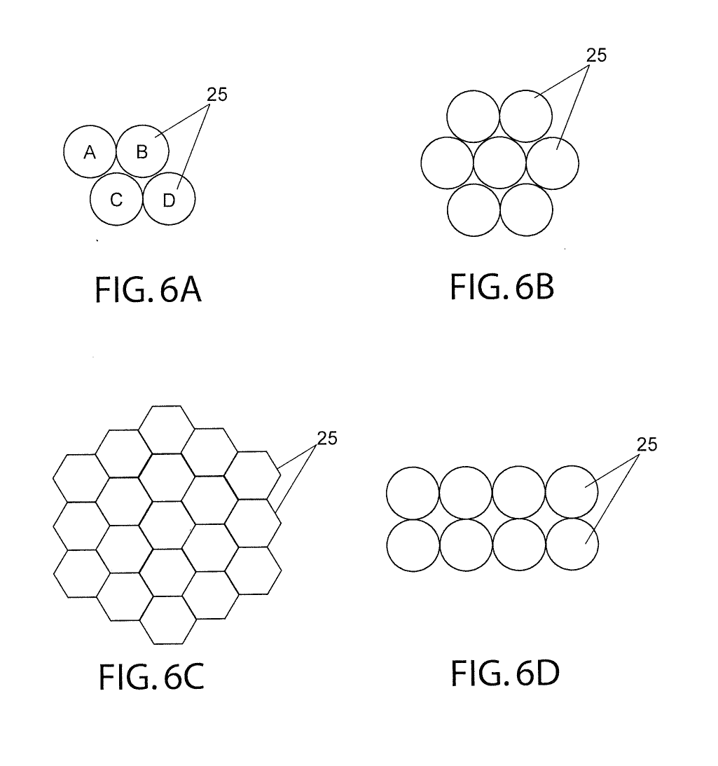

Turning again to FIG. 4, a number of transducers 25 may be provided to allow the location of the blood vessel of interest to be accurately identified and monitored for blood pressure, even at low pressure. For example, between 4 and 10 transducers 25 may be used. In FIGS. 4 and 6A, 4 transducers 25 are shown arranged in a staggered configuration (e.g., each pair of transducers is arranged in a row, with the first transducer of each row offset from the other by half a transducer diameter). FIG. 6B also shows a staggered configuration of transducers 25, with 6 transducers arranged circumferentially about a central transducer. In some cases, more than 10 transducers 25 may be used to achieve the desired configuration and coverage of the blood vessel region. For example, 19 transducers 25 may be arranged in a hexagonal 5×5×5 array, as shown in FIG. 6C.

Linear, non-staggered configurations of transducers 25 may also be used. For example, as shown in FIG. 6D, two rows of 4 transducers 25 may be provided, with one row arranged directly on top of the other. Unlike with the staggered arrangements shown in FIGS. 6A, 6B, and 6C, however, a purely aligned arrangement (as shown in FIG. 6D) may allow for certain areas of the subject's blood vessel region not to be covered by a transducer 25. This may be seen in terms of the amount of white space that results in the configuration of FIG. 6D as compared to the configurations of FIGS. 6A, 6B, and 6C. Other numbers and configurations of transducers 25 are also contemplated, such as 1 row of transducers or 3 or more rows of transducers. In addition, although circular transducers 25 are illustrated in the figures, transducers having other shapes, such as oval, square, or rectangular, may be used. Moreover, the arrangement of transducers 25 may be such as to create an array of transducers, as described above, and the shape of the array may be circular, oval, square, rectangular, hexagonal, or otherwise, as desired. In addition, the array of transducers 25 may, in some embodiments, be attached or otherwise mounted to a contoured and/or flexible material or grid. In this way, the grid may be configured to substantially match the contour of the surface of the body to which it is applied, which may provide for clearer and cleaner transmission and receipt of the signals described above.

By arranging the transducers 25 to cover an area, placement of the sphygmomanometer cuff about the subject's blood vessel area need not precisely align a particular transducer with the targeted blood vessel. In other words, as long as the area covered by the transducers generally overlaps with the blood vessel region having a blood vessel, a return signal should be detectable by one or more of the provided transducers 25, and the systolic blood pressure should be determinable.

In this regard, embodiments of the invention provide an apparatus 50 in which each transducer 25 is configured to transmit a signal toward the blood vessel region, and the apparatus is configured to select one of the transducers corresponding to the highest strength of return signal so as to determine the systolic blood pressure via the selected transducer. For example, with reference to FIG. 6A, transducers A-D may all be directed to transmit an ultrasonic signal in the direction of the subject's blood vessel. In this example, the return signals that are detected may have the following relative strengths:

| Transducer | A | B | C | D |
|---|---|---|---|---|
| Strength | Weak | Average | Strong | Weak |

Because in the above example transducer C detected the strongest return signal as compared to transducers A, D, and to a lesser extent transducer B, transducer C may be selected by the apparatus as being located closest to the blood vessel and, thus, best situated for monitoring the blood pressure of the patient. In this example, transducer C may then continue to send and receive the ultrasonic signals in a near-continuous monitoring scenario, while the other three transducers may be idle. Use of an array of transducers may be particularly helpful for finding the blood vessel in cases in which the subject's blood pressure is very low, which generally causes the blood vessel to be difficult to locate manually.

In other embodiments, the transducers may be configured to act as a phased array for determining a location of the blood vessel. For example, an array of ultrasonic transducers may be configured to operate in a 1 MHz to 20 MHz frequency range, and a single frequency driver may be used for all of the transducers. The relative phases of the respective signals being transmitted by each transducer may be varied (e.g., by the frequency driver) in such a way that the effective pattern of the transmitted signals is reinforced in a desired direction and suppressed in undesired directions. As a result, the transducers may be configured to “sweep” the reinforced transmitted signal over an area to detect a location of the blood vessel. Once the location of the blood vessel is detected, the phased array may focus on that location and use all of the transducers to “listen” for the Korotkoff sound as described above.

In some embodiments, the transducers 25 may comprise piezoelectric crystals. The piezoelectric crystals may be configured to generate a voltage pattern in response to sensing the return signal from the blood vessel, and this voltage pattern may thus correspond to the velocity of the blood flow through the blood vessel. The resulting voltage pattern may be transmitted back to the apparatus to be converted to a sound and/or to be analyzed in some embodiments, such as for graphing trends or determining whether the first Korotkoff sound has been achieved. In other words, the one or more transducers 25 may be used to “listen” for the Korotkoff sound, and the apparatus 50 may be configured to translate the sound into a pressure corresponding to the systolic blood pressure of the subject. In other embodiments, however, other types of transducers may be used, such as ferroelectric materials, polarized ceramics, piezo polymers, and composites.

In some cases, the systolic blood pressure that is detected may be compared against predetermined ranges of blood pressure to determine whether the detected blood pressure falls within a normal range. For example, the apparatus 50 may be configured, in some embodiments, to provide an audible alarm in an instance in which the systolic blood pressure is outside a predetermined range of acceptable values, such as below the lowest value of the predetermined range or above the highest value of the range. The predetermined range may be, for example, approximately 90 mm Hg to approximately 120 mm Hg. Thus, in the event that the detected blood pressure is, for example, 83 mm Hg, the apparatus may provide an audible alarm such that medical personnel may be alerted that the detected blood pressure is outside the range of normal values.

The audible alarm may, for example, be a loud continuous tone, a bell, or some other sound indicating an alarm condition. In some cases, the apparatus 50 may also provide a visual indication of the alarm condition, such as by turning on a light outside the subject's room or a light on a display indicating the subject's room number from among all of the rooms on a floor of a hospital at a nurse's station. In this way, medical personnel can quickly and efficiently attend to the needs of the subject in an attempt to prevent the subject's blood pressure from dropping further.

Similarly, an alarm condition may be created in the event the detected blood pressure is higher than the upper value of the predetermined range of values (such as higher than 120 mm Hg in this example), and a visual and/or audible alarm may likewise be produced.

In some cases, the alarm condition may initiate a change in the blood pressure monitoring scenario, such as to cause the subject's blood pressure to be monitored on a near-continuous basis or to change the frequency of blood pressure readings (e.g., readings every 20 seconds rather than every 5 minutes). In this way, further changes in the subject's blood pressure can be closely monitored and reported back to the appropriate medical personnel.

In some embodiments, the apparatus may provide for amplification and projection of the sound of the blood flow through the blood vessel, such that a doctor or other medical personnel can hear the sound of the blood flow as the blood pressure is automatically being detected and measured. This may assist the doctor in assessing the quality of the subject's blood pressure and/or pulse, for example, to see if the pulse is tri-phasic versus mono- or bi-phasic. Amplification and projection of the blood flow sounds may also allow the doctor to confirm the accuracy of the automatic blood pressure measurements by manually listening for the first Korotkoff sound and comparing the pressure at which the first Korotkoff sound is heard with the blood pressure that is determined by the apparatus.

As noted above, the apparatus 50 may be configured to analyze the blood pressure data obtained from the subject in various ways. Different algorithms may be used to assess the return signals detected by the transducers and translate those signals into blood pressure readings. Moreover, other algorithms may be executed by the apparatus to provide for coordination of the inflation and deflation of the sphygmomanometer cuff, such as for a near-continuous monitoring scenario.

In addition to simply identifying the blood pressure corresponding to the subject's systolic blood pressure as described above, the apparatus 50 may be configured to plot the various blood pressures that are determined over time and may be configured to identify trends in the blood pressure readings. For example, the apparatus 50 may be configured to calculate a slope of the graph of blood pressure over time, such that a sudden drop in blood pressure BP (e.g., ΔBP/time greater than a predetermined amount) may be cause to provide an alarm, even if the blood pressure itself is still within an acceptable range. As another example, the subject's pulse may be charted and compared to a graph of a “healthy” pulse (e.g., by comparing the area under the graphs) to identify any concerns or issues and to facilitate the early detection of illness.

In still other embodiments, the apparatus 50 may be configured to provide an audible indication of a quality of blood flow through a blood vessel, such as to convey to the practitioner whether the subject's condition is stable (e.g., no change in blood flow characteristics, such as blood pressure), improving (e.g., better blood flow characteristics), or declining (e.g., worse blood flow characteristics). For example, a medical device such as the device 20 illustrated in FIG. 1 may be used to measure a subject's blood pressure. In this case, and as described above, the medical device may include a sphygmomanometer cuff 22 that is configured to apply a pressure to a blood vessel region so as to restrict the flow of blood through the blood vessel of the blood vessel region, and a plurality of transducers 25 supported by the sphygmomanometer cuff. As described above, in some embodiments each transducer may be configured to transmit an ultrasonic signal toward the blood vessel region and to detect a return signal indicative of a velocity of blood flow through the blood vessel. The subject's blood pressure may be determined at a detected first Korotkoff sound, for example as described above.

The apparatus may be configured to provide an audible output, such as a sound having a particular pitch and provided at a particular volume (e.g. loudness). The apparatus may be further configured to calibrate the audible output to a velocity of blood flow through the blood vessel at the determined blood pressure. In this way, the audible output may correspond to the velocity of blood flowing through the particular subject's blood vessel at the systolic blood pressure determined by the device 20. The audible output may, for example, comprise a frequency component (corresponding to the pitch perceived by the practitioner) and an intensity component (corresponding to the volume perceived by the practitioner). Calibration of the audible output may comprise configuring the audible output such that the frequency component corresponds to the velocity of the blood flow through the blood vessel and the intensity component corresponds to the determined blood pressure at the detected first Korotkoff sound.

Once the audible output has been calibrated to the subject's blood flow at the first Korotkoff sound, the subject's condition may continue to be monitored without further application of pressure to the blood vessel (e.g., the cuff 22 may be deflated and left in a deflated state). Rather, the velocity of blood flow through the blood vessel may continue to be detected, such as via the transducers 25 of the device 20. A blood pressure at the detected velocity may be extrapolated or otherwise calculated by the apparatus, such as by comparing the detected velocity to the calibrated velocity. In this regard, an algorithm may be applied to calculate the subject's blood pressure based on the detected velocity (e.g., the measured frequency and intensity of the sound produced by the blood flowing through the blood vessel). The algorithm may thus reflect the phenomenon observed by the inventors that as blood pressure decreases, the frequency of the sound produced by the blood flow decreases in a predictable manner and the intensity of the sound produced by the blood flow increases in a predictable manner.

Moreover, in some embodiments, the frequency and intensity components of the audible output may be adjusted in response to the detected velocity through the blood vessel at the extrapolated blood pressure. In this way, a trained practitioner may be able to perceive through the sense of sound alone, e.g., listening to the changes in the audible output, the trends in the condition of the subject, such as whether the subject's condition is improving, declining, or staying the same.

In some embodiments, the continuous monitoring of the patient's blood pressure via the detected velocity may be adjusted and re-calibrated on a periodic basis. For example, the subject's blood pressure may be re-determined at another detected first Korotkoff sound, such as by re-inflating the cuff 22 of the device 20 to “listen” for the first Korotkoff sound. The re-determined blood pressure (at this second instance of the first Korotkoff sound) may then be used to re-calibrate the audible output to a velocity of blood flow through the blood vessel at the re-determined blood pressure. In this way, the extrapolation of a blood pressure at a detected velocity may be compared to the actual measured blood pressure at the same or similar detected velocity, and the audible output conveying the information to the practitioner may be re-calibrated based on the results of the comparison. Moreover, in some cases, the algorithm used to extrapolate the blood pressure from the detected velocity may be adjusted or fine-tuned based on this “second data point” regarding the blood flow velocity to blood pressure relationship.

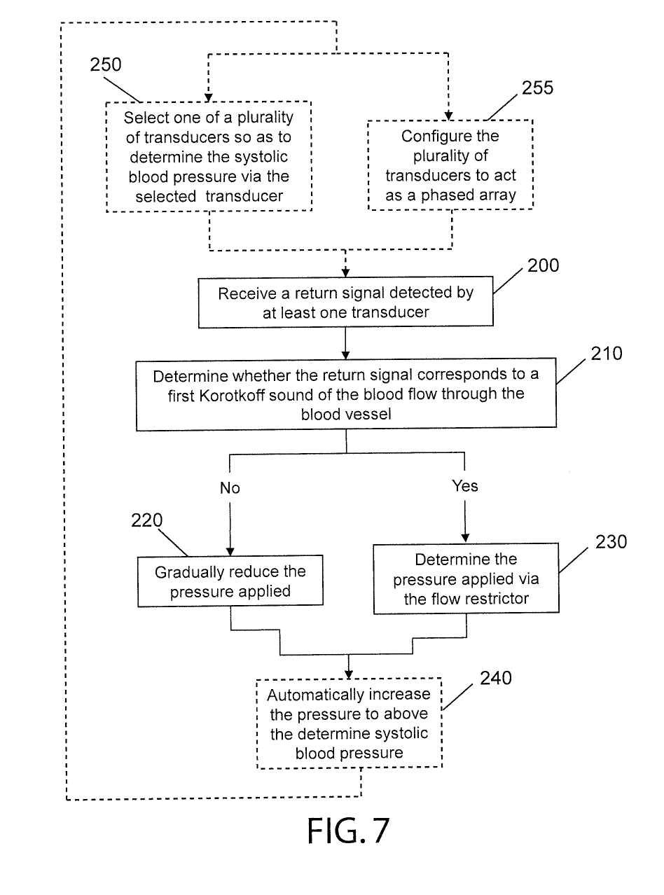

FIG. 7 illustrates flowcharts of systems, methods, and computer program products according to example embodiments of the invention. It will be understood that each block of the flowchart, and combinations of blocks in the flowchart, may be implemented by various means, such as hardware, firmware, processor, circuitry, and/or other devices associated with execution of software including one or more computer program instructions. For example, one or more of the procedures described above may be embodied by computer program instructions. In this regard, the computer program instructions which embody the procedures described above may be stored by a memory device of an apparatus employing an embodiment of the present invention and executed by a processor in the apparatus. As will be appreciated, any such computer program instructions may be loaded onto a computer or other programmable apparatus (e.g., hardware) to produce a machine, such that the resulting computer or other programmable apparatus implements the functions specified in the flowchart block(s). These computer program instructions may also be stored in a computer-readable memory that may direct a computer or other programmable apparatus to function in a particular manner, such that the instructions stored in the computer-readable memory produce an article of manufacture the execution of which implements the function specified in the flowchart block(s). The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart block(s).

Accordingly, blocks of the flowchart support combinations of means for performing the specified functions, combinations of operations for performing the specified functions, and program instruction means for performing the specified functions. It will also be understood that one or more blocks of the flowchart, and combinations of blocks in the flowchart, can be implemented by special purpose hardware-based computer systems which perform the specified functions, or combinations of special purpose hardware and computer instructions.

Turning to FIG. 7, embodiments of a method for monitoring blood pressure are provided that include the steps of receiving a return signal detected by at least one transducer, wherein the return signal is indicative of a velocity of blood flow through a blood vessel at Block 200. Next, a processor may be used to determine whether the return signal corresponds to a first Korotkoff sound of the blood flow through the blood vessel at Block 210. The pressure applied via a sphygmomanometer cuff disposed proximate a subject's blood vessel region may be gradually reduced in an instance in which the return signal does not correspond to the first Korotkoff sound (Block 220), whereas the pressure applied via the cuff may be determined in an instance in which the return signal corresponds to the first Korotkoff sound (Block 230). The pressure determined by the apparatus may thus be considered the systolic blood pressure of the subject, as described above.

In some cases, the pressure applied via the sphygmomanometer cuff may be automatically increased to a pressure above the determined systolic blood pressure and gradually reduced to re-determine the systolic blood pressure in a near-continuous blood pressure monitoring scenario. Block 240. Moreover, the transducers may be used to determine a location of the blood vessel. In some embodiments, one of a plurality of transducers corresponding to the highest strength of return signal may be selected so as to determine the systolic blood pressure via the selected transducer. Block 250. In other embodiments, the plurality of transducers may be configured to act as a phased array for determining a location of the blood vessel. Block 255.

In some embodiments, certain ones of the operations above may be modified or further amplified as described below. Furthermore, in some embodiments, additional optional operations may be included, examples of which are shown in dashed lines in FIG. 7. Modifications, additions, or amplifications to the operations above may be performed in any order and in any combination.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A system for measuring a subject's blood pressure comprising:
a sphygmomanometer cuff configured to apply a pressure to a blood vessel region so as to restrict the flow of blood through a blood vessel of the blood vessel region;
at least one transducer configured to transmit a signal toward the blood vessel region and to detect a return signal indicative of a velocity of blood flow through the blood vessel; and
an apparatus comprising a processor configured to:
receive the return signal detected by the transducer,
determine whether the return signal corresponds to a first Korotkoff sound of the blood flow through the blood vessel,
gradually reduce the pressure applied via the sphygmomanometer cuff in an instance in which the return signal does not correspond to the first Korotkoff sound, and
determine the pressure applied via the sphygmomanometer cuff in an instance in which the return signal corresponds to the first Korotkoff sound,
wherein the pressure determined by the apparatus is a systolic blood pressure of the subject.

2. The system of claim 1, wherein the apparatus is configured to automatically increase the pressure applied via the sphygmomanometer cuff to a pressure above the determined systolic blood pressure and gradually reduce the pressure applied via the sphygmomanometer cuff to re-determine the systolic blood pressure in a near-continuous blood pressure monitoring scenario.

3. The system of claim 1, wherein the system comprises a plurality of transducers arranged in a staggered configuration.

4. The system of claim 3, wherein the apparatus is configured to select one of the transducers corresponding to the highest strength of return signal so as to determine the systolic blood pressure via the selected transducer.

5. The system of claim 3, wherein the plurality of transducers is configured to act as a phased array for determining a location of the blood vessel.

6. The system of claim 1, wherein the apparatus further comprises a display, wherein the apparatus is configured to present upon the display a graphical representation of the systolic blood pressure determined.

7. The system of claim 1, wherein the apparatus is configured to provide an audible alarm in an instance in which the systolic blood pressure is outside a predetermined range of acceptable values.

8. A medical device for measuring a subject's blood pressure comprising:
a sphygmomanometer cuff configured to apply a pressure to a blood vessel region so as to restrict the flow of blood through a blood vessel of the blood vessel region; and
a plurality of transducers supported by the sphygmomanometer cuff, wherein each transducer is configured to transmit an ultrasonic signal toward the blood vessel region and to detect a return signal indicative of a velocity of blood flow through the blood vessel,
wherein the return signal detected by each transducer is conveyed to an apparatus so as to allow a pressure applied via the sphygmomanometer cuff corresponding to a first Korotkoff sound detected via at least one of the plurality of transducers to be determined as a systolic blood pressure of the subject.

9. The medical device of claim 8, wherein the plurality of transducers comprises between 4 transducers and 10 transducers.

10. The medical device of claim 8, wherein the plurality of transducers are arranged in a staggered configuration.

11. The medical device of claim 8, wherein the plurality of transducers comprises piezoelectric crystals.

12. The medical device of claim 8, wherein the ultrasonic signal has a frequency of between approximately 4 MHz and approximately 20 MHz.

13. The medical device of claim 8, wherein the plurality of transducers is configured to act as a phased array for determining a location of the blood vessel.

14-23. (canceled)

24. A method of measuring a subject's blood pressure comprising:
receiving a return signal detected by at least one transducer, wherein the return signal is indicative of a velocity of blood flow through a blood vessel,
determining, via a processor, whether the return signal corresponds to a first Korotkoff sound of the blood flow through the blood vessel,
gradually reducing the pressure applied via a sphygmomanometer cuff disposed proximate a subject's blood vessel region in an instance in which the return signal does not correspond to the first Korotkoff sound, and
determining the pressure applied via the sphygmomanometer cuff in an instance in which the return signal corresponds to the first Korotkoff sound,
wherein the pressure determined by the apparatus is a systolic blood pressure of the subject.

25. The method of claim 24 further comprising automatically increasing the pressure applied via the sphygmomanometer cuff to a pressure above the determined systolic blood pressure and gradually reducing the pressure applied via the sphygmomanometer cuff to re-determine the systolic blood pressure in a near-continuous blood pressure monitoring scenario.

26. The method of claim 24 further comprising selecting one of a plurality of transducers corresponding to the highest strength of return signal so as to determine the systolic blood pressure via the selected transducer.

27. The method of claim 24 further comprising configuring the plurality of transducers to act as a phased array for determining a location of the blood vessel.

28. A method of providing an audible indication of a quality of blood flow through a blood vessel comprising:
determining a subject's blood pressure at a detected first Korotkoff sound;
calibrating an audible output to a velocity of blood flow through the blood vessel at the determined blood pressure, wherein the audible output comprises a frequency component corresponding to the velocity of the blood flow through the blood vessel and an intensity component corresponding to the determined blood pressure at the detected first Korotkoff sound;
detecting the velocity of blood flow through the blood vessel;
extrapolating a blood pressure at the detected velocity; and
adjusting the frequency and intensity components of the audible output in response to the detected velocity through the blood vessel at the extrapolated blood pressure.

29. The method of claim 28 further comprising:
re-determining the subject's blood pressure at another detected first Korotkoff sound; and
re-calibrating the audible output to a velocity of blood flow through the blood vessel at the re-determined blood pressure.

30. The method of claim 28, wherein the first Korotkoff sound is detected using a medical device comprising:
a sphygmomanometer cuff configured to apply a pressure to a blood vessel region so as to restrict the flow of blood through the blood vessel of the blood vessel region; and
a plurality of transducers supported by the sphygmomanometer cuff, wherein each transducer is configured to transmit an ultrasonic signal toward the blood vessel region and to detect a return signal indicative of a velocity of blood flow through the blood vessel.
